(19) United States
(12) Patent Application Publication
(10) Pub. No.: US 2021/0228557 A1
(43) Pub. Date: Jul. 29, 2021

(54) THERAPEUTIC COMPOSITIONS COMPRISING COENZYME Q10

(71) Applicant: Healthy Directions, LLC, Bethesda, MD (US)

(72) Inventor: Stephen SINATRA, Charlotte, NC (US)

(73) Assignee: Healthy Directions, LLC, Bethesda, MD (US)

(21) Appl. No.: 17/160,785

(22) Filed: Jan. 28, 2021

Related U.S. Application Data
(60) Provisional application No. 62/967,243, filed on Jan. 29, 2020.

(51) Int. Cl. A61K 31/4415; A61K 31/714; A61K 31/519; A61K 33/00; A61K 31/202; A61K 31/205; A61K 31/122

(52) U.S. Cl. CPC A61K 31/4415; A61K 31/714; A61K 31/519; A61K 31/05; A61K 31/202; A61K 31/205; A61K 31/122; A61K 33/00

(57) ABSTRACT

Disclosed herein are therapeutic compositions comprising omega-3 fatty acid mixtures and ubiquinone, and a method improving heart health comprising administering an effective amount of a therapeutic composition comprising omega-3 fatty acid mixtures and ubiquinone. According to embodiments, therapeutic compositions for improved heart health are provided comprising: vitamin B6; folate; vitamin B12; chromium; an omega-3 fatty acid component, comprising: docosahexaenoic acid (DHA), and eicosapentaenoic acid (EPA), and L-carnitine fumarate; and ubiquinone. The therapeutic compositions are effective in supporting heart health and healthy aging.

### RELATED APPLICATIONS

This application claims priority to U.S. Provisional Patent Application No. 62/967,243, filed Jan. 29, 2020, the contents of which are incorporated herein by reference in their entirety.

### BACKGROUND

Cardiovascular disease is the leading cause of death for both men and women. What is needed is a natural and safe therapeutic composition that provides support for heart health, cellular energy, and overall wellness.

### SUMMARY

According to embodiments, therapeutic compositions are provided, comprising omega-3 fatty acid mixtures, Coenzyme Q10 (ubiquinone), and cardiovascular support ingredients, and a method of increasing cardiovascular health is provided comprising administering an effective amount of a therapeutic composition comprising omega-3 fatty acid mixtures, Coenzyme Q10 (ubiquinone), and cardiovascular support ingredients.

A therapeutic composition for heart health is provided comprising: vitamin B6; folate; vitamin B12; chromium; an omega-3 fatty acid component, comprising: docosahexaenoic acid (DHA), and eicosapentaenoic acid (EPA), and L-carnitine fumarate; and ubiquinone.

A therapeutic composition for heart health is provided comprising: vitamin B6; folate; vitamin B12; chromium; an omega-3 fatty acid component, comprising: docosahexaenoic acid (DHA), and eicosapentaenoic acid (EPA), and L-carnitine fumarate; ubiquinone; and trans resveratrol.

A therapeutic composition for heart health is provided comprising: vitamin B6; folate; vitamin B12; chromium; an omega-3 fatty acid component, comprising: docosahexaenoic acid (DHA), and eicosapentaenoic acid (EPA), and L-carnitine fumarate; ubiquinone; trans resveratrol; and turmeric extract.

A method for improving heart health is provided, comprising administering to an individual an effective dose of a composition comprising: vitamin B6; folate; vitamin B12; chromium; an omega-3 fatty acid component, comprising: docosahexaenoic acid (DHA), and eicosapentaenoic acid (EPA), and L-carnitine fumarate; and ubiquinone.

A method for improving heart health is provided, comprising administering to an individual an effective dose of a composition comprising: vitamin B6; folate; vitamin B12; chromium; an omega-3 fatty acid component, comprising: docosahexaenoic acid (DHA), and eicosapentaenoic acid (EPA), and L-carnitine fumarate; ubiquinone; and trans resveratrol.

A method for improving heart health is provided, comprising administering to an individual an effective dose of a composition comprising: vitamin B6; folate; vitamin B12; chromium; an omega-3 fatty acid component, comprising: docosahexaenoic acid (DHA), and eicosapentaenoic acid (EPA), and L-carnitine fumarate; ubiquinone; trans resveratrol; and turmeric extract.

### DETAILED DESCRIPTION

Described herein are therapeutic compositions comprising omega-3 fatty acid mixtures, ubiquinone, and other cardiovascular support ingredients, and methods of improving heart health comprising administering an effective amount of a therapeutic composition comprising omega-3 fatty acid mixtures, ubiquinone, and cardiovascular support ingredients.

In one aspect, described herein a therapeutic composition comprising: an omega-3 fatty acid component, and ubiquinone.

In some embodiments, the amount of omega-3 fatty acid component is about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg.

According to embodiments, the omega-3 fatty acid component comprises docosahexaenoic acid (DHA) and eicosapentaenoic acid (EPA). In some embodiments, the amount of DHA in the omega-3 fatty acid component is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg. According to embodiments, the amount of EPA in the omega-3 fatty acid component is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

In some embodiments, the omega-3 fatty acid component comprises an oil concentrate from squid. According to embodiments, the squid oil may be, for example CalaMarine® Oil Concentrate. In some embodiments, the amount of squid oil concentrate is about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg.

In some embodiments, the amount of DHA in the squid oil concentrate is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg. According to embodiments, the amount of EPA in the squid oil concentrate is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

In some embodiments, the omega-3 fatty acid component comprises an oil concentrate from fish and/or from algal and/or hill. In some embodiments, the amount of fish and/or algal and/or krill oil concentrate is about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg.

In some embodiments, the amount of DHA in the oil concentrate from fish and/or from algal and/or krill is about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg. According to embodiments, the amount of EPA in the oil concentrate from fish and/or from algal and/or krill is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

In some embodiments, the therapeutic composition includes an omega-7 fatty acid component comprising an oil concentrate from palmitoleic acid. In some embodiments, the amount of oil concentrate from palmitoleic acid is about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg.

According to embodiments, the ubiquinone, also known as Coenzyme Q10, may be, for example Hydro Q-Sorb®. In some embodiments, the amount of Coenzyme Q10 is about 10mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg.

According to some embodiments, the Coenzyme Q10, may be, for example from MicroActive CoQ10 and/or Ubiquisome CoQ10 and/or Vesi-sorb CoQ10 and/or Kaneka QH and/or unbranded varieties of ubiquinone and/or unbranded varieties of ubiquinol. In some embodiments, the amount of Coenzyme Q10 is about 10mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg.

According to embodiments, the composition may contain other ingredients that support cardiovascular, heart, brain function, general wellness, or aging. In some embodiment, the support ingredients are from a natural source. In some embodiments, the support ingredients are synthetic. In some embodiments, the support ingredients are a combination of natural source and synthetic. In some embodiments, the support ingredients may include vitamin B6, folate, vitamin B12, chromium, L-carnitine fumarate, trans resveratrol, turmeric extract, and combinations thereof.

According to embodiments, the vitamin B6 may be pyridoxine HCL, pyridoxal-5-phosphate. In some embodiments, the amount of vitamin B6 is 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg.

In some embodiments, the amount of folate is about 100 mcg dietary folate equivalents (DFE), about 200 mcg DFE, about 300 mcg DFE, about 400 mcg DFE, about 500 mcg DFE, about 600 mcg DFE, about 700 mcg DFE, about 800 mcg DFE, about 900 mcg DFE, about 1000 mcg DFE, about 1100 mcg DFE, about 1200 mcg DFE, about 1300 mcg DFE, about 1400 mcg DFE, or about 1500 mcg DFE. In some embodiments, the amount of folate is about 50 mcg folic acid, about 100 mcg folic acid, about 150 mcg folic acid, about 200 mcg folic acid, about 250 mcg folic acid, about 300 mcg folic acid, about 350 mcg folic acid, about 400 mcg folic acid, about 450 mcg folic acid, about 500 mcg folic acid, about 550 mcg folic acid, about 600 mcg folic acid, about 650 mcg folic acid, about 700 mcg folic acid, about 750 mcg folic acid, about 800 mcg folic acid, about 850 mcg folic acid, about 900 mcg folic acid, about 950 mcg folic acid, or about 1000 mcg folic acid.

According to embodiments, the chromium may be, for example, Crominex® 3+ chromium stabilized with Capros® standardized Amla Extract (fruit), and/or PrimaVie® purified and standardized Shilajit, and/or from Polynictinate, and/or from Picolinate. In some embodiments, the amount of chromium is about 50mcg, about 100 mcg, about 150 mcg, about 200 mcg, about 250 mcg, about 300 mcg, about 350 mcg, about 400 mcg, about 450 mcg, about 500 mcg, about 550 mcg, about 600 mcg, about 650 mcg, about 700 mcg, about 750 mcg, about 800 mcg, about 850 mcg, about 900 mcg, about 950 mcg, or about 1000 mcg.

In some embodiments, the amount of L-carnitine fumarate is about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490, or about 500 mg.

In some embodiments, the amount of trans resveratrol is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg.

According to embodiments, the turmeric extract (rhizome) may be, for example, CurcuWlN®, and/or HydroCurc®, and/or with Curcuminoid. In some embodiments, the amount of turmeric extract is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg.

According to embodiments, the composition may include an excipient. In some embodiments, the excipient may be a coloring agent. In some embodiments, the excipient may be a diluent. In some embodiments, the excipient may be a binder. In some embodiments, the excipient may be a granulating agent. In some embodiments, the excipient may be a bulking agent. In some embodiments, the excipient may be a disintegrant. In some embodiments, the excipient may be a glidant. In some embodiments, the excipient may be a flavorant. In some embodiments, the excipient may be a preservative. In some embodiments, the excipient may be a stabilizer. In some embodiments, the excipient may be lipophilic, polymeric, cellulosic or combinations of any thereof. In some embodiments, the excipient may be at least one of gelatin, glycerin, gamma-cyclo-dextrin, medium chain triglycerides, yellow beeswax, purified water, sunflower lecithin, mixed tocopherols, annatto, sunflower oil, mannitol, silica, lemon oil, microcrystalline cellulose, hypromellose, ascorbyl palminate, povidone, or a combination thereof.

In yet another aspect, described herein is a method of improving heart health and promoting general wellness comprising administering to an individual an effect dose of a composition comprising vitamin B6; folate; vitamin B12; chromium; an omega-3 fatty acid component, comprising: docosahexaenoic acid (DHA), and eicosapentaenoic acid (EPA), and L-carnitine fumarate; and ubiquinone. According to embodiments, the composition comprises vitamin B6; folate; vitamin B12; chromium; an omega-3 fatty acid component, comprising: docosahexaenoic acid (DHA), and eicosapentaenoic acid (EPA), and L-carnitine fumarate; ubiquinone, and trans resveratrol. According to embodiments, the composition comprises vitamin B6; folate; vitamin B12; chromium; an omega-3 fatty acid component, comprising: docosahexaenoic acid (DHA), and eicosapentaenoic acid (EPA), and L-carnitine fumarate; ubiquinone; transrsveratrol; and turmeric extract.

According to embodiments, the composition may be administered orally. According to embodiments, the composition may be encapsulated.

According to embodiments, the therapeutic composition comprises vitamin B6; folate; vitamin B12; chromium; an omega-3 fatty acid component, comprising: docosahexaenoic acid (DHA), and eicosapentaenoic acid (EPA), and L-carnitine fumarate; and ubiquinone.

According to embodiments, the therapeutic composition comprises vitamin B6; folate; vitamin B12; chromium; an omega-3 fatty acid component, comprising: docosahexaenoic acid (DHA), and eicosapentaenoic acid (EPA), and L-carnitine fumarate; ubiquinone; and trans resveratrol.

According to embodiments, the therapeutic composition comprises vitamin B6; folate; vitamin B12; chromium; an omega-3 fatty acid component, comprising: docosahexaenoic acid (DHA), and eicosapentaenoic acid (EPA), and L-carnitine fumarate; ubiquinone; and turmeric extract.

According to embodiments, the therapeutic composition comprises vitamin B6; folate; vitamin B12; chromium; an omega-3 fatty acid component, comprising: docosahexaenoic acid (DHA), and eicosapentaenoic acid (EPA), and L-carnitine fumarate; ubiquinone; trans resveratrol; and turmeric extract.

According to embodiments, the therapeutic composition comprises vitamin B6; folate; vitamin B12; chromium; an omega-3 fatty acid component, comprising: docosahexaenoic acid (DHA), and eicosapentaenoic acid (EPA), and L-carnitine fumarate; and ubiquinone and further comprises at least one excipient. According to embodiments, the excipient may be at least one of gelatin, glycerin, gamma-cyclo-dextrin, medium chain triglycerides, yellow beeswax, purified water, sunflower lecithin, mixed tocopherols, annatto, sunflower oil, mannitol, silica, lemon oil, microcrystalline cellulose, hypromellose, ascorbyl palminate, povidone, or a combination thereof. According to embodiments, the excipient may be gelatin, glycerin, gamma-cyclo-dextrin, medium chain triglycerides, yellow beeswax, purified water, sunflower lecithin, mixed tocopherols, annatto, sunflower oil, mannitol, silica, lemon oil, and microcrystalline cellulose, or a combination thereof. According to embodiments, the excipient may be gelatin, glycerin, gamma-cyclo-dextrin, povidone, medium chain triglycerides, yellow beeswax, purified water, sunflower lecithin, mixed tocopherols, annatto, sunflower oil, hypromellose, mannitol, silica, lemon oil, microcrystalline cellulose, and ascorbyl palminate, or a combination thereof.

According to embodiments, the omega-3 fatty acid component comprises squid oil.

According to embodiments, the therapeutic composition comprises at least 10 mg vitamin B6; at least 100 mcg dietary equivalents (DFE) folate; at least 100 mcg vitamin B12; at least 50 mcg chromium; at least 100 mg omega-3 fatty acid component, comprising: at least 50 mg DHA, and at least 25 mg EPA, and at least 10 mg L-carnitine fumarate; and at least 10 mg ubiquinone.

According to embodiments, the therapeutic composition comprises at least 10 mg vitamin B6; at least 100 mcg dietary equivalents (DFE) folate; at least 100 mcg vitamin B12; at least 50 mcg chromium; at least 100 mg omega-3 fatty acid component, comprising: at least 50 mg DHA, and at least 25 mg EPA, and at least 10 mg L-carnitine fumarate; at least 10 mg ubiquinone; and at least at least 1 mg trans resveratrol.

According to embodiments, the therapeutic composition comprises at least 10 mg vitamin B6; at least 100 mcg dietary equivalents (DFE) folate; at least 100 mcg vitamin B12; at least 50 mcg chromium; at least 100 mg omega-3 fatty acid component, comprising: at least 50 mg DHA, and at least 25 mg EPA, and at least 10 mg L-carnitine fumarate; at least 10 mg ubiquinone; and at least 25 mg turmeric extract.

According to embodiments, the therapeutic composition comprises at least 10 mg vitamin B6; at least 100 mcg dietary equivalents (DFE) folate; at least 100 mcg vitamin B12; at least 50 mcg chromium; at least 100 mg omega-3 fatty acid component, comprising: at least 50 mg DHA, and at least 25 mg EPA, and at least 10 mg L-carnitine fumarate; at least 10 mg ubiquinone; at least at least 1 mg trans resveratrol; and at least 25 mg turmeric extract.

According to embodiments, the omega-3 fatty acid component comprises squid oil.

According to embodiments, the therapeutic composition comprises from about 10 mg to about 100 mg vitamin B6; from about 100 mcg DFE to about 1500 mcg DFE folate; from about 100 mcg to about 2000 mcg vitamin B12; from about 50 mcg to about 1000 chromium; from about 100 mg to about 30000 omega-3 fatty acid component, comprising: from about 50 mg to about 1000 mg DHA, and from about 25 mg to about 500 mg EPA, and from about 10 mg to about 500 mg L-carnitine fumarate; and from about 10 mg to about 300 mg ubiquinone.

According to embodiments, the therapeutic composition comprises from about 10 mg to about 100 mg vitamin B6; from about 100 mcg DFE to about 1500 mcg DFE folate; from about 100 mcg to about 2000 mcg vitamin B12; from about 50 mcg to about 1000 chromium; from about 100 mg to about 30000 omega-3 fatty acid component, comprising: from about 50 mg to about 1000 mg DHA, and from about 25 mg to about 500 mg EPA, and from about 10 mg to about 500 mg L-carnitine fumarate; and from about 10 mg to about 300 mg ubiquinone; and from about 1 mg to about 100 mg trans resveratrol.

According to embodiments, the therapeutic composition comprises from about 10 mg to about 100 mg vitamin B6; from about 100 mcg DFE to about 1500 mcg DFE folate; from about 100 mcg to about 2000 mcg vitamin B12; from about 50 mcg to about 1000 chromium; from about 100 mg to about 30000 omega-3 fatty acid component, comprising: from about 50 mg to about 1000 mg DHA, and from about 25 mg to about 500 mg EPA, and from about 10 mg to about 500 mg L-carnitine fumarate; and from about 10 mg to about 300 mg ubiquinone; and from about 25 mg to about 1000 mg turmeric extract.

According to embodiments, the therapeutic composition comprises from about 10 mg to about 100 mg vitamin B6; from about 100 mcg DFE to about 1500 mcg DFE folate; from about 100 mcg to about 2000 mcg vitamin B12; from about 50 mcg to about 1000 chromium; from about 100 mg to about 30000 omega-3 fatty acid component, comprising: from about 50 mg to about 1000 mg DHA, and from about 25 mg to about 500 mg EPA, and from about 10 mg to about 500 mg L-carnitine fumarate; and from about 10 mg to about 300 mg ubiquinone; from about 1 mg to about 100 mg trans resveratrol; and from about 25 mg to about 1000 mg turmeric extract.

According to embodiments, the therapeutic composition comprises about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; and about 50 mg ubiquinone.

According to embodiments, the therapeutic composition comprises about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 50 mg ubiquinone; and about 30 mg trans resveratrol.

According to embodiments, the therapeutic composition comprises about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 50 mg ubiquinone; and about 250 mg turmeric extract.

According to embodiments, the therapeutic composition comprises about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 50 mg ubiquinone; about 30 mg trans resveratrol; and about 250 mg turmeric extract.

According to embodiments, the therapeutic composition comprises about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; and about 100 mg ubiquinone.

According to embodiments, the therapeutic composition comprises about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 100 mg ubiquinone; and about 30 mg trans resveratrol.

According to embodiments, the therapeutic composition comprises about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 100 mg ubiquinone; and about 250 mg turmeric extract.

According to embodiments, the therapeutic composition comprises about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 100 mg ubiquinone; about 30 mg trans resveratrol; and about 250 mg turmeric extract.

According to embodiments, the therapeutic composition comprises at least 10 mg vitamin B6; at least 100 mcg dietary equivalents (DFE) folate; at least 100 mcg vitamin B12; at least 50 mcg chromium; at least 100 mg omega-3 fatty acid component, comprising: at least 50 mg DHA, and at least 25 mg EPA, and at least 10 mg L-carnitine fumarate; and at least 10 mg ubiquinone. In some embodiment, the therapeutic composition also comprises between about 500 mcg to about 50 mg of Astaxanthin, and/or between about 50 mg to about 200 mg of magnesium, and/or between about 45 mcg to about 360 mcg of Vitamin K2 (as MK7), and/or between about 50 IU to about 5000 IU of Vitamin D, and/or between about 10 mg to about 1000 mg of Vitamin C, and/or between about 25 IU to about 800 IU of Vitamin E, and/or between about 10 mg to about 1000 mg of tocotrienols, and/or between about 50 mcg to about 400 mcg of selenium, and/or between about 6 mg to about 40 mg of lutein, and/or between about 1 mg to about 15 mg of lycopene, and/or between about 1 mg to about 10 mg of zeaxanthin, and/or between about 400 mg to about 2000 mg of berberine, and/or between about 1mg to about 100 mg of olive extract, and/or between about 1 mg to about 100 mg of hydroxytyrosol (synthetic or from olive) and/or between about 50 mg to about 500 mg from green tea leaf extract, and/or between about 50 mg to about 2500 mg of magnesium threonate, and/or between about 250 mg to about 2000 mg of citrus bergamot, and/or between about 50 mg to about 300 mg of grape seed extract, and/or between about 50 mg to about 1000 mg of ashwaghanda, and/or olive oil.

According to embodiments, the therapeutic composition is in a capsule.

According to embodiments, a method for improving heart health comprises administering to an individual an effective dose of a composition comprising: vitamin B6; folate; vitamin B12; chromium; an omega-3 fatty acid component, comprising: docosahexaenoic acid (DHA), and eicosapentaenoic acid (EPA), and L-carnitine fumarate; and ubiquinone.

According to embodiments, a method for improving heart health comprises administering to an individual an effective dose of a composition comprising: vitamin B6; folate; vitamin B12; chromium; an omega-3 fatty acid component, comprising: docosahexaenoic acid (DHA), and eicosapentaenoic acid (EPA), and L-carnitine fumarate; ubiquinone; and trans resveratrol.

According to embodiments, a method for improving heart health comprises administering to an individual an effective dose of a composition comprising: vitamin B6; folate; vitamin B12; chromium; an omega-3 fatty acid component, comprising: docosahexaenoic acid (DHA), and eicosapentaenoic acid (EPA), and L-carnitine fumarate; ubiquinone; trans resveratrol; and turmeric extract.

According to embodiments, a method for improving heart health comprises administering to an individual an effective dose of a composition comprising about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; and about 50 mg ubiquinone.

According to embodiments, a method for improving heart health comprises administering to an individual an effective dose of a composition comprising about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 50 mg ubiquinone; and about 30 mg trans resveratrol.

According to embodiments, a method for improving heart health comprises administering to an individual an effective dose of a composition comprising about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 50 mg ubiquinone; about 30 mg trans resveratrol; and about 250 mg turmeric extract.

According to embodiments, a method for improving heart health comprises administering to an individual an effective dose of a composition comprising about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; and about 100 mg ubiquinone.

According to embodiments, a method for improving heart health comprises administering to an individual an effective dose of a composition comprising about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 100 mg ubiquinone; and about 30 mg trans resveratrol.

According to embodiments, a method for improving heart health comprises administering to an individual an effective dose of a composition comprising about 30 mg vitamin B6; about 667 mcg DFE folate; about 750 mcg vitamin B12; about 400 mcg chromium; about 1250 mg omega-3 fatty acid component, comprising: about 350 mg DHA, and about 200 mg EPA, and about 174 mg L-carnitine fumarate; about 100 mg ubiquinone; about 30 mg trans resveratrol; and about 250 mg turmeric extract.

### EXAMPLES

The following examples are provided for illustrative purposes only, and are intended to be purely exemplary of the disclosure and are not intended to limit the scope of the claims provided herein. Efforts have been made to ensure accuracy with respect to numbers (e.g., amounts, temperature, etc.), but some errors and deviations should be accounted for.

### Example 1

| | Amount Per Serving | | |
|---|---|---|---|
| | Dosage | | % DV |
| Active Ingredients | | | |
| Vitamin B6 (as pyridoxine HCl, pyridoxal-5- | 30 | mg | 1765% |
| phosphate) | | | |
| Folate | 667 | mcg | 167% |
| | (400 mcg | DFE | |
| | folic acid) | | |
| Vitamin B12 (as cyanocobalamin) | 750 | mcg | 31250% |
| Chromium (as Crominex ® 3+ chromium | 400 | mcg | 1143% |
| stabilized with Capros ® standardized Amla | | | |
| Extract (fruit), PrimaVie ® purified and | | | |
| standardized Shilajit) | | | |
| CalaMarine ® Oil Concentrate (from squid) | 1250 | mg | |
| DHA (docosahexaenoic acid) | 350 | mg | |
| EPA (eicosapentaenoic acid) | 200 | mg | |
| L-Carnitine Fumarate | 174 | mg | |
| Coenzyme Q10 (as ubiquinone) | 50 | mg | |
| (Hydro Q-Sorb ® ) | | | |

Other Ingredients
Gelatin, glycerin, gamma-cyclo-dextrin, medium chain
triglycerides, yellow beeswax, purified water, sunflower lecithin,
mixed tocopherols (preservative), annatto (color) suspension in
sunflower oil, mannitol, silica, lemon oil, microcrystalline cellulose.

### Example 2

| | Amount Per Serving | | |
|---|---|---|---|
| | Dosage | | % DV |
| Active Ingredients | | | |
| Vitamin B6 (as pyridoxine HCl, pyridoxal-5- | 30 | mg | 1765% |
| phosphate) | | | |
| Folate | 667 | mcg | 167% |
| | (400 mcg | DFE | |
| | folic acid) | | |
| Vitamin B12 (as cyanocobalamin) | 750 | mcg | 31250% |
| Chromium (as Crominex ® 3+ chromium | 400 | mcg | 1143% |
| stabilized with Capros ® standardized Amla | | | |
| Extract (fruit), PrimaVie ® purified and | | | |
| standardized Shilajit) | | | |
| CalaMarine ® Oil Concentrate (from squid) | 1250 | mg | |
| DHA (docosahexaenoic acid) | 350 | mg | |
| EPA (eicosapentaenoic acid) | 200 | mg | |
| L-Carnitine Fumarate | 174 | mg | |
| Coenzyme Q10 (as ubiquinone) | 100 | mg | |
| (Hydro Q-Sorb ® ) | | | |

Other Ingredients
Gelatin, glycerin, gamma-cyclo-dextrin, medium chain
triglycerides, yellow beeswax, purified water, sunflower lecithin,
mixed tocopherols (preservative), annatto (color) suspension in
sunflower oil, mannitol, silica, lemon oil, microcrystalline cellulose.

### Example 3

| | Amount Per Serving | | |
|---|---|---|---|
| | Dosage | | % DV |
| Active Ingredients | | | |
| Vitamin B6 (as pyridoxine HCl, pyridoxal-5- | 30 | mg | 1765% |
| phosphate) | | | |
| Folate | 667 | mcg | 167% |
| | (400 mcg | DFE | |
| | folic acid) | | |
| Vitamin B12 (as cyanocobalamin) | 750 | mcg | 31250% |
| Chromium (as Crominex ® 3+ chromium | 400 | mcg | 1143% |
| stabilized with Capros ® standardized Amla | | | |
| Extract (fruit), PrimaVie ® purified and | | | |
| standardized Shilajit) | | | |
| CalaMarine ® Oil Concentrate (from squid) | 1250 | mg | |
| DHA (docosahexaenoic acid) | 350 | mg | |
| EPA (eicosapentaenoic acid) | 200 | mg | |
| L-Carnitine Fumarate | 174 | mg | |
| Coenzyme Q10 (as ubiquinone) | 50 | mg | |
| (Hydro Q-Sorb ® ) | | | |
| Trans Resveratrol | 30 | mg | |

Other Ingredients
Gelatin, glycerin, gamma-cyclo-dextrin, medium chain
triglycerides, yellow beeswax, purified water, sunflower lecithin,
mixed tocopherols (preservative), annatto (color) suspension in
sunflower oil, mannitol, silica, lemon oil, microcrystalline cellulose.

### Example 4

| | Amount Per Serving | | |
|---|---|---|---|
| | Dosage | | % DV |
| Active Ingredients | | | |
| Vitamin B6 (as pyridoxine HCl, pyridoxal-5- | 30 | mg | 1765% |
| phosphate) | | | |
| Folate | 667 | mcg | 167% |
| | (400 mcg | DFE | |
| | folic acid) | | |
| Vitamin B12 (as cyanocobalamin) | 750 | mcg | 31250% |
| Chromium (as Crominex ® 3+ chromium | 400 | mcg | 1143% |
| stabilized with Capros ® standardized Amla | | | |
| Extract (fruit), PrimaVie ® purified and | | | |
| standardized Shilajit) | | | |
| CalaMarine ® Oil Concentrate (from squid) | 1250 | mg | |
| DHA (docosahexaenoic acid) | 350 | mg | |
| EPA (eicosapentaenoic acid) | 200 | mg | |
| L-Carnitine Fumarate | 174 | mg | |
| Coenzyme Q10 (as ubiquinone) | 100 | mg | |
| (Hydro Q-Sorb ® ) | | | |
| Trans Resveratrol | 30 | mg | |

Other Ingredients
Gelatin, glycerin, gamma-cyclo-dextrin, medium chain
triglycerides, yellow beeswax, purified water, sunflower lecithin,
mixed tocopherols (preservative), annatto (color) suspension in
sunflower oil, mannitol, silica, lemon oil, microcrystalline cellulose.

### Example 5

| | Amount Per Serving | | |
|---|---|---|---|
| | Dosage | | % DV |
| Active Ingredients | | | |
| Vitamin B6 (as pyridoxine HCl, pyridoxal-5- | 30 | mg | 1765% |
| phosphate) | | | |
| Folate | 667 | mcg | 167% |
| | (400 mcg | DFE | |
| | folic acid) | | |
| Vitamin B12 (as cyanocobalamin) | 750 | mcg | 31250% |
| Chromium (as Crominex ® 3+ chromium | 400 | mcg | 1143% |
| stabilized with Capros ® standardized Amla | | | |
| Extract (fruit), PrimaVie ® purified and | | | |
| standardized Shilajit) | | | |
| CalaMarine ® Oil Concentrate (from squid) | 1250 | mg | |
| DHA (docosahexaenoic acid) | 350 | mg | |
| EPA (eicosapentaenoic acid) | 200 | mg | |
| Turmeric Extract (rhizome) CurcuWIN ® ) | 250 | mg | |
| L-Carnitine Fumarate | 174 | mg | |
| Coenzyme Q10 (as ubiquinone) | 50 | mg | |
| (Hydro Q-Sorb ® ) | | | |
| Trans Resveratrol | 30 | mg | |

Other Ingredients
Gelatin, glycerin, gamma-cyclo-dextrin, medium chain
triglycerides, yellow beeswax, purified water, sunflower lecithin,
mixed tocopherols (preservative), annatto (color) suspension in
sunflower oil, mannitol, silica, lemon oil, microcrystalline cellulose.

### Example 6

| | Amount Per Serving | | |
|---|---|---|---|
| | Dosage | | % DV |
| Active Ingredients | | | |
| Vitamin B6 (as pyridoxine HCl, pyridoxal-5- | 30 | mg | 1765% |
| phosphate) | | | |
| Folate | 667 | mcg | 167% |
| | (400 mcg | DFE | |
| | folic acid) | | |
| Vitamin B12 (as cyanocobalamin) | 750 | mcg | 31250% |
| Chromium (as Crominex ® 3+ chromium | 400 | mcg | 1143% |
| stabilized with Capros ® standardized Amla | | | |
| Extract (fruit), PrimaVie ® purified and | | | |
| standardized Shilajit) | | | |
| CalaMarine ® Oil Concentrate (from squid) | 1250 | mg | |
| DHA (docosahexaenoic acid) | 350 | mg | |
| EPA (eicosapentaenoic acid) | 200 | mg | |
| Turmeric Extract (rhizome) CurcuWIN ® ) | 250 | mg | |
| L-Carnitine Fumarate | 174 | mg | |
| Coenzyme Q10 (as ubiquinone) | 100 | mg | |
| (Hydro Q-Sorb ® ) | | | |
| Trans Resveratrol | 30 | mg | |

Other Ingredients
Gelatin, glycerin, gamma-cyclo-dextrin, medium chain
triglycerides, yellow beeswax, purified water, sunflower lecithin,
mixed tocopherols (preservative), annatto (color) suspension in
sunflower oil, mannitol, silica, lemon oil, microcrystalline cellulose.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A therapeutic composition for heart health comprising:
vitamin B6;
folate;
vitamin B 12;
chromium;
an omega-3 fatty acid component, comprising:
docosahexaenoic acid (DHA), and
eicosapentaenoic acid (EPA), and L-carnitine fumarate; and
ubiquinone.

2. The therapeutic composition of claim 1, further comprising:
trans resveratrol.

3. The therapeutic composition of claim 1, further comprising:
turmeric extract.

4. The therapeutic composition of claim 1, further comprising:
trans resveratrol; and
turmeric extract.

5. The therapeutic composition of claim 1, further comprising at least one excipient.

6. The therapeutic composition of claim 5, wherein the excipient is at least one of gelatin, glycerin, gamma-cyclo-dextrin, medium chain triglycerides, yellow beeswax, purified water, sunflower lecithin, mixed tocopherols, annatto, sunflower oil, mannitol, silica, lemon oil, microcrystalline cellulose, hypromellose, ascorbyl palminate, povidone, or a combination thereof.

7. The therapeutic composition according to claim 6, comprising: gelatin, glycerin, gamma-cyclo-dextrin, medium chain triglycerides, yellow beeswax, purified water, sunflower lecithin, mixed tocopherols, annatto, sunflower oil, mannitol, silica, lemon oil, and microcrystalline cellulose.

8. The therapeutic composition of claim 6, comprising: gelatin, glycerin, gamma-cyclo-dextrin, povidone, medium chain triglycerides, yellow beeswax, purified water, sunflower lecithin, mixed tocopherols, annatto, sunflower oil, hypromellose, mannitol, silica, lemon oil, microcrystalline cellulose, and ascorbyl palminate.

9. The therapeutic composition of claim 1, wherein the omega-3 fatty acid component comprises squid oil.

10. The composition of claim 1, comprising:
at least 10 mg vitamin B6;
at least 100 mcg dietary equivalents (DFE) folate;
at least 100 mcg vitamin B12;
at least 50 mcg chromium;
at least 100 mg omega-3 fatty acid component, comprising:
at least 50 mg DHA, and
at least 25 mg EPA, and
at least 10 mg L-carnitine fumarate; and
at least 10 mg ubiquinone.

11. The therapeutic composition of claim 10, further comprising at least at least 10 mg trans resveratrol.

12. The therapeutic composition of claim 10, further comprising at least 25 mg turmeric extract.

13. The therapeutic composition of claim 10, wherein the omega-3 fatty acid component comprises squid oil.

14. The therapeutic composition of claim 10, further comprising:
at least at least 1 mg trans resveratrol; and
at least 25 mg turmeric extract.

15. The therapeutic composition of claim 1, comprising:
from about 10 mg to about 100 mg vitamin B6;
from about 100 mcg DFE to about 1500 mcg DFE folate;
from about 100 mcg to about 2000 mcg vitamin B12;
from about 50 mcg to about 1000 chromium;
from about 100 mg to about 30000 omega-3 fatty acid component, comprising:
from about 50 mg to about 1000 mg DHA, and
from about 25 mg to about 500 mg EPA, and
from about 10 mg to about 500 mg L-carnitine fumarate; and
from about 10 mg to about 300 mg ubiquinone.

16. The composition of claim 15, further comprising from about 1 mg to about 100 mg trans resveratrol.

17. The composition of claim 15, further comprising from about 25 mg to about 1000 mg turmeric extract.

18. The composition of claim 15, further comprising:
from about 1 mg to about 100 mg trans resveratrol; and
from about 25 mg to about 1000 mg turmeric extract.

19. The composition of claim 1, comprising:
about 30 mg vitamin B6;
about 667 mcg DFE folate;
about 750 mcg vitamin B12;
about 400 mcg chromium;
about 1250 mg omega-3 fatty acid component, comprising:
about 350 mg DHA, and
about 200 mg EPA, and
about 174 mg L-carnitine fumarate; and
about 50 mg ubiquinone.

20. The composition of claim 19, further comprising about 30 mg trans resveratrol.

21. The composition of claim 19, further comprising about 250 mg turmeric extract.

22. The composition of claim 19, further comprising:
about 30 mg trans resveratrol; and
about 250 mg turmeric extract.

23. The composition of claim 1, comprising:
about 30 mg vitamin B6;
about 667 mcg DFE folate;
about 750 mcg vitamin B12;
about 400 mcg chromium;
about 1250 mg omega-3 fatty acid component, comprising:
about 350 mg DHA, and
about 200 mg EPA, and
about 174 mg L-carnitine fumarate; and
about 100 mg ubiquinone.

24. The composition of claim 23, further comprising about 30 mg trans resveratrol.

25. The composition of claim 23, further comprising about 250 mg turmeric extract.

26. The composition of claim 23, further comprising:
about 30 mg trans resveratrol; and
about 250 mg turmeric extract.

27. The composition of claim 1 in a capsule.

28. A method for improving heart health, comprising administering to an individual an effective dose of a composition comprising:
vitamin B6;
folate;
vitamin B12;
chromium;
an omega-3 fatty acid component, comprising:
docosahexaenoic acid (DHA), and
eicosapentaenoic acid (EPA), and
L-carnitine fumarate; and
ubiquinone.

29. The method of claim 28, wherein the composition further comprises trans resveratrol.

30. The method of claim 28, wherein the composition further comprises:
trans resveratrol; and
turmeric extract.

31. The method of claim 28, wherein the composition comprises:
about 30 mg vitamin B6;
about 667 mcg DFE folate;
about 750 mcg vitamin B12;
about 400 mcg chromium;
about 1250 mg omega-3 fatty acid component, comprising:
about 350 mg DHA, and
about 200 mg EPA, and
about 174 mg L-carnitine fumarate; and
about 50 mg ubiquinone.

32. The method of claim 31, wherein the composition further comprises about 10 mg trans resveratrol.

33. The method of claim 31, wherein the composition further comprises:
about 30 mg trans resveratrol; and
about 250 mg turmeric extract.

34. The method of claim 33, wherein the composition comprises:
about 30 mg vitamin B6;
about 667 mcg DFE folate;
about 750 mcg vitamin B12;
about 400 mcg chromium;
about 1250 mg omega-3 fatty acid component, comprising:
about 350 mg DHA, and
about 200 mg EPA, and
about 174 mg L-carnitine fumarate; and
about 100 mg ubiquinone.

35. The method of claim 34, wherein the composition further comprises about 30 mg trans resveratrol.

36. The method of claim 34, wherein the composition further comprises:
about 30 mg trans resveratrol; and
about 250 mg turmeric extract.
